(19) United States
(12) Patent Application Publication
(10) Pub. No.: US 2019/0099579 A1
(43) Pub. Date: Apr. 4, 2019

(54) PRESSURE GENERATING DEVICE AND AIR FILTER HAVING A CORRELATED ARRANGEMENT OF MAGNETS

(71) Applicant: KONINKLIJKE PHILIPS N.V., Eindhoven (NL)

(72) Inventors: Christopher Randall BAKER, North Huntington, PA (US); Mark William DIMATTEO, Irwin, PA (US); Ryan GRANT, Pittsburgh, PA (US)

(21) Appl. No.: 16/142,710

(22) Filed: Sep. 26, 2018

Related U.S. Application Data
(60) Provisional application No. 62/565,163, filed on Sep. 29, 2017.

(51) Int. Cl. A61M 16/10; B01D 46/00; A61M 16/00; A61M 16/08

(52) U.S. Cl. CPC A61M 16/107; B01D 46/0005; H01F 7/02; A61M 16/0816; A61M 16/0063

(57) ABSTRACT

An air filtration assembly includes a filter portion formed from a suitable filtration media and a mounting portion disposed adjacent the filter portion. The mounting portion includes a series of first magnetic field emission structures positioned therein or thereon.

### CROSS-REFERENCE TO PRIOR APPLICATIONS

This application claims the benefit of U.S. Provisional Application No. 62/565163, filed on 29 Sep. 2017. This application is hereby incorporated by reference herein.

### BACKGROUND OF THE INVENTION

### 1. Field of the Invention

The present invention pertains to pressure generating devices and air filtration assemblies therefor. More particularly, the present invention pertains to pressure generating devices and air filtration assemblies which utilize magnetic elements. The present invention also pertains to systems for generating a flow of gas.

### 2. Description of the Related Art

Many individuals suffer from disordered breathing during sleep. Sleep apnea is a common example of such sleep disordered breathing suffered by millions of people throughout the world. One type of sleep apnea is obstructive sleep apnea (OSA), which is a condition in which sleep is repeatedly interrupted by an inability to breathe due to an obstruction of the airway; typically the upper airway or pharyngeal area. Obstruction of the airway is generally believed to be due, at least in part, to a general relaxation of the muscles which stabilize the upper airway segment, thereby allowing the tissues to collapse the airway. Another type of sleep apnea syndrome is a central apnea, which is a cessation of respiration due to the absence of respiratory signals from the brain's respiratory center. An apnea condition, whether obstructive, central, or mixed, which is a combination of obstructive and central, is defined as the complete or near cessation of breathing, for example a 90% or greater reduction in peak respiratory air-flow.

Those afflicted with sleep apnea experience sleep fragmentation and complete or nearly complete cessation of ventilation intermittently during sleep with potentially severe degrees of oxyhemoglobin desaturation. These symptoms may be translated clinically into extreme daytime sleepiness, cardiac arrhythmias, pulmonary-artery hypertension, congestive heart failure and/or cognitive dysfunction. Other consequences of sleep apnea include right ventricular dysfunction, carbon dioxide retention during wakefulness, as well as during sleep, and continuous reduced arterial oxygen tension. Sleep apnea sufferers may be at risk for excessive mortality from these factors as well as by an elevated risk for accidents while driving and/or operating potentially dangerous equipment.

Even if a patient does not suffer from a complete or nearly complete obstruction of the airway, it is also known that adverse effects, such as arousals from sleep, can occur where there is only a partial obstruction of the airway. Partial obstruction of the airway typically results in shallow breathing referred to as a hypopnea. A hypopnea is typically defined as a 50% or greater reduction in the peak respiratory air-flow. Other types of sleep disordered breathing include, without limitation, upper airway resistance syndrome (UARS) and vibration of the airway, such as vibration of the pharyngeal wall, commonly referred to as snoring.

It is well known to treat sleep disordered breathing by applying a continuous positive air pressure (CPAP) to the patient's airway. This positive pressure effectively “splints” the airway, thereby maintaining an open passage to the lungs. It is also known to provide a positive pressure therapy in which the pressure of gas delivered to the patient varies with the patient's breathing cycle, or varies with the patient's breathing effort, to increase the comfort to the patient. This pressure support technique is referred to as bi-level pressure support, in which the inspiratory positive airway pressure (IPAP) delivered to the patient is higher than the expiratory positive airway pressure (EPAP). It is further known to provide a positive pressure therapy in which the pressure is automatically adjusted based on the detected conditions of the patient, such as whether the patient is experiencing an apnea and/or hypopnea. This pressure support technique is referred to as an auto-titration type of pressure support, because the pressure support device seeks to provide a pressure to the patient that is only as high as necessary to treat the disordered breathing.

Pressure support therapies as just described involve the placement of a patient interface device including a mask component having a soft, flexible sealing cushion on the face of the patient. The mask component may be, without limitation, a nasal mask that covers the patient's nose, a nasal/oral mask that covers the patient's nose and mouth, or a full face mask that covers the patient's face. Such patient interface devices may also employ other patient contacting components, such as forehead supports, cheek pads and chin pads. The patient interface device is typically secured to the patient's head by a headgear component. The patient interface device is connected to a gas delivery tube or conduit and interfaces the pressure support device with the airway of the patient, so that a flow of breathing gas can be delivered from the pressure/flow generating device to the airway of the patient.

CPAPs and ventilators used in pressure support therapies use air filters or air filtration assemblies to remove airborne solid particles from the air such as dust, pollen, mold and bacteria. This ensures that the solid particles do not reach the patient's respiratory system. Air impedance characteristics of these filters are specified for each device by the original equipment manufacturer. Using the approved filter ensures that the patient is protected from both airborne solid particles and given the desired therapy. It can be difficult for a user to know if the filter is installed properly.

### SUMMARY OF THE INVENTION

As one aspect of the invention, an air filtration assembly comprises: a filter portion formed from a suitable filtration media and a mounting portion disposed adjacent the filter portion, the mounting portion having a series of first magnetic field emission structures positioned therein or thereon.

The series of first magnetic field emission structures may be in the form of a ferrous magnetic strip.

The first magnetic field emission structures may be coupled to the mounting portion via over-molding.

The first magnetic field emission structures may be coupled to the mounting portion via heat staking.

The first magnetic field emission structures may be coupled to the mounting portion via ultrasonic welding.

The mounting portion may be formed from a magnetic plastic which includes the first magnetic field emission structures.

The mounting portion may comprise a compliant seal structured and positioned to sealingly engage against another object.

The mounting portion may be formed generally as a frame positioned around the filter portion.

As another aspect of the present invention, a pressure generating device for producing a flow of a gas comprises: a housing; an air compressor disposed in the housing for producing the flow of the gas; and an air inlet defined in the housing, the air inlet in communication with the air compressor and structured to allow the passage of air therethrough to the air compressor for creating the flow of the gas. The housing comprises a series of second magnetic field emission structures positioned therein or thereon at or about the air inlet, the series of second magnetic field emission structures positioned and structured to interact with a corresponding first series of magnetic field emission structures associated with a filtration assembly which is disposable at the air inlet.

The series of second magnetic field emission structures may be in the form of a ferrous magnetic strip.

As yet another aspect, a system for generating a flow of a gas comprises a pressure generating device comprising: a housing; an air compressor disposed in the housing for producing the flow of the gas; and an air inlet defined in the housing, the air inlet in communication with the air compressor and structured to allow the passage of air therethrough to the air compressor for creating the flow of the gas. The system also comprises an air filtration assembly coupled to the housing at or about the air inlet. The air filtration assembly comprises: a filter portion formed from a suitable filtration media; and a mounting portion disposed adjacent the filter portion, the mounting portion having a series of first magnetic field emission structures positioned therein or thereon, the first magnetic field structures oriented according to a code. The housing comprises a series of second magnetic field emission structures positioned therein or thereon at or about the air inlet, the second magnetic field structures oriented according to a mirror image of the code, and the filtration assembly is coupled to the housing by the magnetic attraction between the first magnetic field emission structures and the second magnetic field emission structures.

The series of first magnetic field emission structures may be in the form of a ferrous magnetic strip and the series of second magnetic field emission structures may be in the form of another ferrous magnetic strip.

The system may further comprise a compliant seal disposed between the mounting portion and the housing of the pressure generating device.

The mounting portion of the filtration element may comprise a compliant seal sealingly engaged against the housing of the pressure generating device.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIGS. 1, 2A-2C, 3A, 3B and 4A-4C are various diagrams used to help explain different concepts about correlated magnetic technology which can be utilized in an embodiment of the present invention;

FIG. 5 is a simplified diagram of an airway pressure support system according to an exemplary embodiment which is operated within an environment, such as a bedroom or home of the user of airway pressure support system, shown with a patient interface device thereof disposed on the face of a patient;

FIG. 6 is a schematic view of an air filtration assembly in accordance with an example embodiment of the present invention;

FIG. 7 is a sectional view of the air filtration assembly of FIG. 6 taken along line 7-7 of FIG. 6;

FIG. 8 is a schematic isometric view of a pressure generating device in accordance with an example embodiment of the present invention;

FIG. 9 is a sectional view of the pressure generating device of FIG. 8 taken along line 9-9 of FIG. 8;

FIG. 10 is a schematic isometric view of a system for generating a flow of breathing gas including a pressure generating device and an air filtration assembly installed thereon in accordance with an example embodiment of the present invention; and

FIG. 11 is a sectional view of the system of FIG. 10 taken along line 11-11 of FIG. 10.

### DETAILED DESCRIPTION OF EXEMPLARY EMBODIMENTS

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As used herein, the statement that two or more parts or components “engage” one another shall mean that the parts exert a force against one another either directly or through one or more intermediate parts or components. As used herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

Embodiments of the present invention are directed generally to filter arrangements which utilize correlated magnetics to promote sealing of the filter to related components. Such arrangements also provide for protection against counterfeit air filters. This significant improvement over the state-of-art is attributable, in part, to the use of an correlated magnetics.

Correlated magnetics was first fully described and enabled in U.S. patent application Ser. No. 12/123,718 filed on May 20, 2008 and entitled “A Field Emission System and Method”, the contents of which are hereby incorporated herein by reference. A second generation of a correlated magnetic technology is described and enabled in U.S. patent application Ser. No. 12/358,423 filed on Jan. 23, 2009 and entitled “A Field Emission System and Method”, the contents of which are hereby incorporated herein by reference. A third generation of a correlated magnetic technology is described and enabled in U.S. patent application Ser. No. 12/476,952 filed on Jun. 2, 2009 and entitled “A Field Emission System and Method”, the contents of which are hereby incorporated herein by reference. Another technology known as correlated inductance, which is related to correlated magnetics, has been described and enabled in U.S. patent application Ser. No. 12/322,561 filed on Feb. 4, 2009 and entitled “A System and Method for Producing and Electric Pulse”. A brief discussion about correlated magnetics is provided first before a detailed discussion is provided about the correlated magnetic mask of the present invention.

### Correlated Magnetics Technology

This section is provided to introduce the reader to basic magnets and correlated magnetic technology. This section includes subsections relating to basic magnets and correlated magnets. It should be understood that this section is provided to assist the reader with understanding the present invention, and should not be used to limit the scope of the present invention.

### A. Magnets

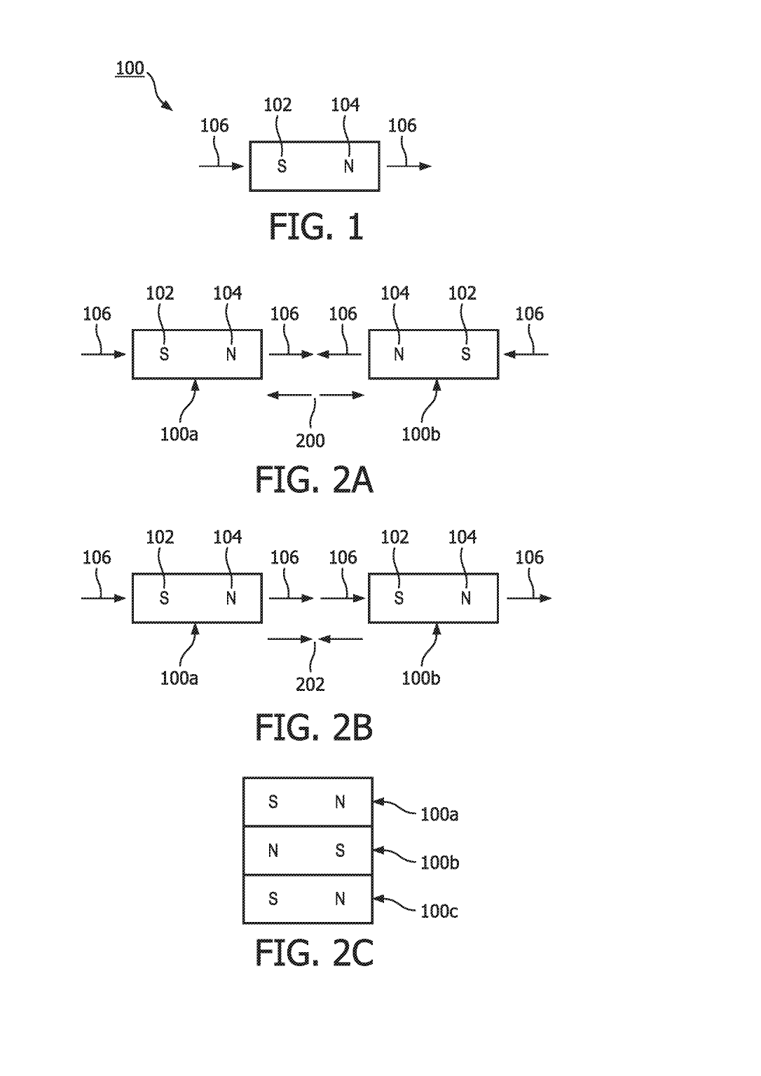

A magnet is a material or object that produces a magnetic field which is a vector field that has a direction and a magnitude (also called strength). Referring to FIG. 1, there is illustrated an exemplary magnet 100 which has a South pole 102 and a North pole 104 and magnetic field vectors 106 that represent the direction and magnitude of the magnet's moment. The magnet's moment is a vector that characterizes the overall magnetic properties of the magnet 100. For a bar magnet, the direction of the magnetic moment points from the South pole 102 to the North pole 104. The North and South poles 104 and 102 are also referred to herein as positive (+) and negative (−) poles, respectively.

Referring to FIG. 2A, there is a diagram that depicts two magnets 100a and 100b aligned such that their polarities are opposite in direction resulting in a repelling spatial force 200 which causes the two magnets 100a and 100b to repel each other. In contrast, FIG. 2B is a diagram that depicts two magnets 100a and 100b aligned such that their polarities are in the same direction resulting in an attracting spatial force 202 which causes the two magnets 100a and 100b to attract each other. In FIG. 2B, the magnets 100a and 100b are shown as being aligned with one another but they can also be partially aligned with one another where they could still “stick” to each other and maintain their positions relative to each other. FIG. 2C is a diagram that illustrates how magnets 100a, 100b and 100c will naturally stack on one another such that their poles alternate.

### B. Correlated Magnets

Correlated magnets can be created in a wide variety of ways depending on the particular application as described in the aforementioned U.S. patent application Ser. Nos. 12/123,718, 12/358,432, and 12/476,952 by using a unique combination of magnet arrays (referred to herein as magnetic field emission sources), correlation theory (commonly associated with probability theory and statistics) and coding theory (commonly associated with communication systems). A brief discussion is provided next to explain how these widely diverse technologies are used in a unique and novel way to create correlated magnets.

Basically, correlated magnets are made from a combination of magnetic (or electric) field emission sources which have been configured in accordance with a pre-selected code having desirable correlation properties. Thus, when a magnetic field emission structure is brought into alignment with a complementary, or mirror image, magnetic field emission structure the various magnetic field emission sources will all align causing a peak spatial attraction force to be produced, while the misalignment of the magnetic field emission structures cause the various magnetic field emission sources to substantially cancel each other out in a manner that is a function of the particular code used to design the two magnetic field emission structures. In contrast, when a magnetic field emission structure is brought into alignment with a duplicate magnetic field emission structure then the various magnetic field emission sources all align causing a peak spatial repelling force to be produced, while the misalignment of the magnetic field emission structures causes the various magnetic field emission sources to substantially cancel each other out in a manner that is a function of the particular code used to design the two magnetic field emission structures.

The aforementioned spatial forces (attraction, repelling) have a magnitude that is a function of the relative alignment of two magnetic field emission structures and their corresponding spatial force (or correlation) function, the spacing (or distance) between the two magnetic field emission structures, and the magnetic field strengths and polarities of the various sources making up the two magnetic field emission structures. The spatial force functions can be used to achieve precision alignment and precision positioning not possible with basic magnets. Moreover, the spatial force functions can enable the precise control of magnetic fields and associated spatial forces thereby enabling new forms of attachment devices for attaching objects with precise alignment and new systems and methods for controlling precision movement of objects. An additional unique characteristic associated with correlated magnets relates to the situation where the various magnetic field sources making-up two magnetic field emission structures can effectively cancel out each other when they are brought out of alignment which is described herein as a release force. This release force is a direct result of the particular correlation coding used to configure the magnetic field emission structures.

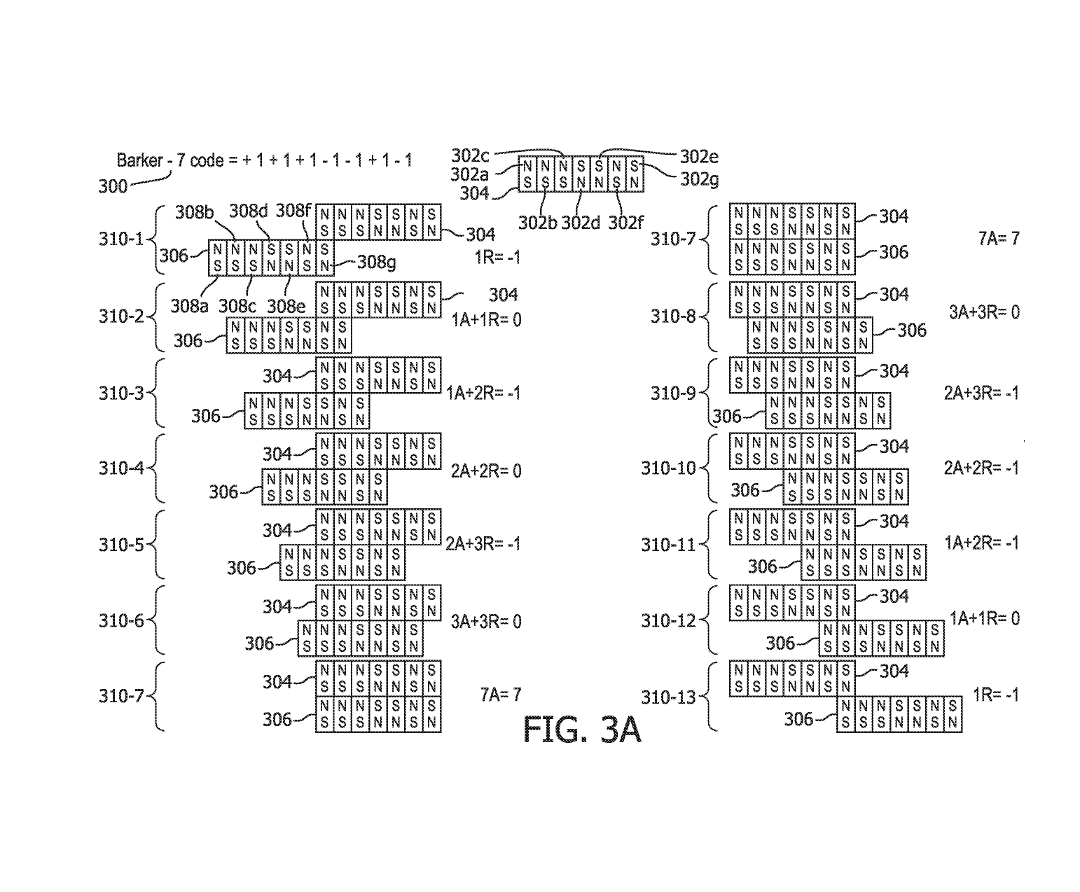

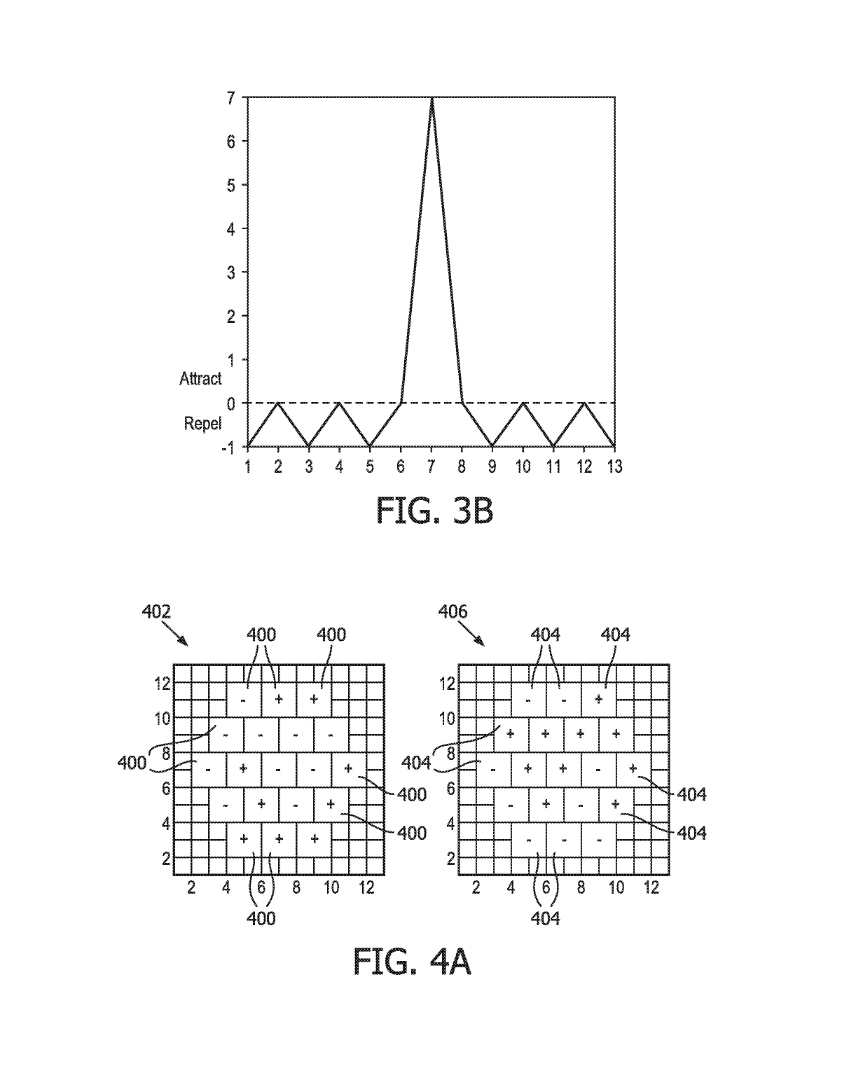

A person skilled in the art of coding theory will recognize that there are many different types of codes that have different correlation properties which have been used in communications for channelization purposes, energy spreading, modulation, and other purposes. Many of the basic characteristics of such codes make them applicable for use in producing the magnetic field emission structures described herein. For example, Barker codes are known for their autocorrelation properties and can be used to help configure correlated magnets. Although, a Barker code is used in an example below with respect to FIGS. 3A-3B, other forms of codes which may or may not be well known in the art are also applicable to correlated magnets because of their autocorrelation, cross-correlation, or other properties including, for example, Gold codes, Kasami sequences, hyperbolic congruential codes, quadratic congruential codes, linear congruential codes, Welch-Costas array codes, Golomb-Costas array codes, pseudorandom codes, chaotic codes, Optimal Golomb Ruler codes, deterministic codes, designed codes, one dimensional codes, two dimensional codes, three dimensional codes, or four dimensional codes, combinations thereof, and so forth.

Referring to FIG. 3A, there are diagrams used to explain how a Barker length 7 code 300 can be used to determine polarities and positions of magnets 302a, 302b . . . 302g making up a first magnetic field emission structure 304. Each magnet 302a, 302b . . . 302g has the same or substantially the same magnetic field strength (or amplitude), which for the sake of this example is provided as a unit of 1 (where A=Attract, R=Repel, A=−R, A=1, R=−1). A second magnetic field emission structure 306 (including magnets 308a, 308b . . . 308g) that is identical to the first magnetic field emission structure 304 is shown in 13 different alignments 310-1 through 310-13 relative to the first magnetic field emission structure 304. For each relative alignment, the number of magnets that repel plus the number of magnets that attract is calculated, where each alignment has a spatial force in accordance with a spatial force function based upon the correlation function and magnetic field strengths of the magnets 302a, 302b . . . 302g and 308a, 308b . . . 308g. With the specific Barker code used, the spatial force varies from −1 to 7, where the peak occurs when the two magnetic field emission structures 304 and 306 are aligned which occurs when their respective codes are aligned. The off peak spatial force, referred to as a side lobe force, varies from 0 to −1. As such, the spatial force function causes the magnetic field emission structures 304 and 306 to generally repel each other unless they are aligned such that each of their magnets are correlated with a complementary magnet (i.e., a magnet's South pole aligns with another magnet's North pole, or vice versa). In other words, the two magnetic field emission structures 304 and 306 substantially correlate with one another when they are aligned to substantially mirror each other.

In FIG. 3B, there is a plot that depicts the spatial force function of the two magnetic field emission structures 304 and 306 which results from the binary autocorrelation function of the Barker length 7 code 300, where the values at each alignment position 1 through 13 correspond to the spatial force values that were calculated for the thirteen alignment positions 310-1 through 310-13 between the two magnetic field emission structures 304 and 306 depicted in FIG. 3A. As the true autocorrelation function for correlated magnet field structures is repulsive, and most of the uses envisioned will have attractive correlation peaks, the usage of the term ‘autocorrelation’ herein will refer to complementary correlation unless otherwise stated. That is, the interacting faces of two such correlated magnetic field emission structures 304 and 306 will be complementary to (i.e., mirror images of) each other. This complementary autocorrelation relationship can be seen in FIG. 3A where the bottom face of the first magnetic field emission structure 304 having the pattern ‘S S S N N S N’ is shown interacting with the top face of the second magnetic field emission structure 306 having the pattern ‘N N N S S N S’, which is the mirror image (pattern) of the bottom face of the first magnetic field emission structure 304.

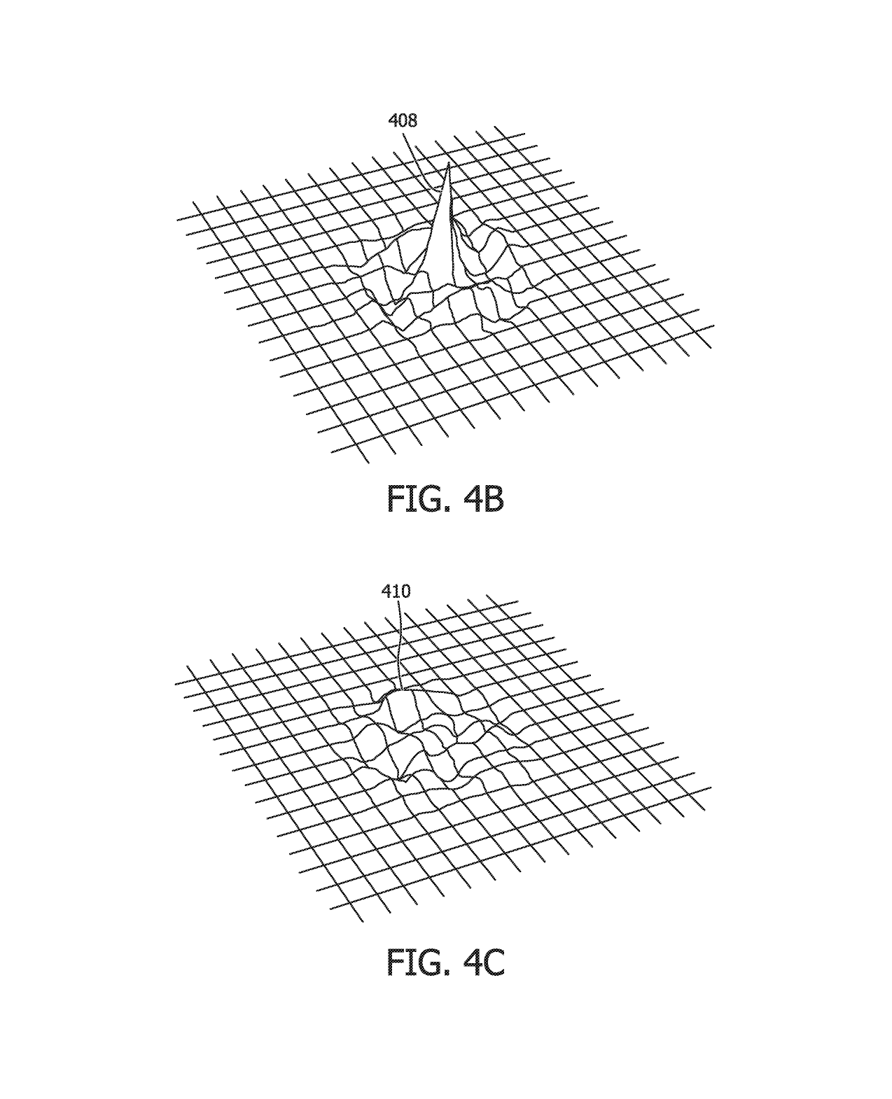

Referring to FIG. 4A, there is a diagram of an array of 19 magnets 400 positioned in accordance with an exemplary code to produce an exemplary magnetic field emission structure 402 and another array of 19 magnets 404 which is used to produce a mirror image magnetic field emission structure 406. In this example, the exemplary code was intended to produce the first magnetic field emission structure 402 to have a first stronger lock when aligned with its mirror image magnetic field emission structure 406 and a second weaker lock when it is rotated 90° relative to its mirror image magnetic field emission structure 406. FIG. 4B depicts a spatial force function 408 of the magnetic field emission structure 402 interacting with its mirror image magnetic field to emission structure 406 to produce the first stronger lock. As can be seen, the spatial force function 408 has a peak which occurs when the two magnetic field emission structures 402 and 406 are substantially aligned. FIG. 4C depicts a spatial force function 410 of the magnetic field emission structure 402 interacting with its mirror magnetic field emission structure 406 after being rotated 90°. As can be seen, the spatial force function 410 has a smaller peak which occurs when the two magnetic field emission structures 402 and 406 are substantially aligned but one structure is rotated 90°. If the two magnetic field emission structures 402 and 406 are in other positions, then they could be easily separated.

In the above examples, the correlated magnets 304, 306, 402 and 406 overcome the normal ‘magnet orientation’ behavior with the aid of a holding mechanism such as an adhesive, a screw, a bolt & nut, etc. In other cases, magnets of the same magnetic field emission structure could be sparsely separated from other magnets (e.g., in a sparse array) such that the magnetic forces of the individual magnets do not substantially interact, in which case the polarity of individual magnets can be varied in accordance with a code without requiring a holding mechanism to prevent magnetic forces from ‘flipping’ a magnet. However, magnets are typically close enough to one another such that their magnetic forces would substantially interact to cause at least one of them to ‘flip’ so that their moment vectors align but these magnets can be made to remain in a desired orientation by use of a holding mechanism such as an adhesive, a screw, a bolt & nut, etc. As such, correlated magnets often utilize some sort of holding mechanism to form different magnetic field emission structures which can be used in a wide-variety of applications like, for example, a turning mechanism, a tool insertion slot, alignment marks, a latch mechanism, a pivot mechanism, a swivel mechanism, a lever, a drill head assembly, a hole cutting tool assembly, a machine press tool, a gripping apparatus, a slip ring mechanism, and a structural assembly.

### Correlated Filter Arrangement

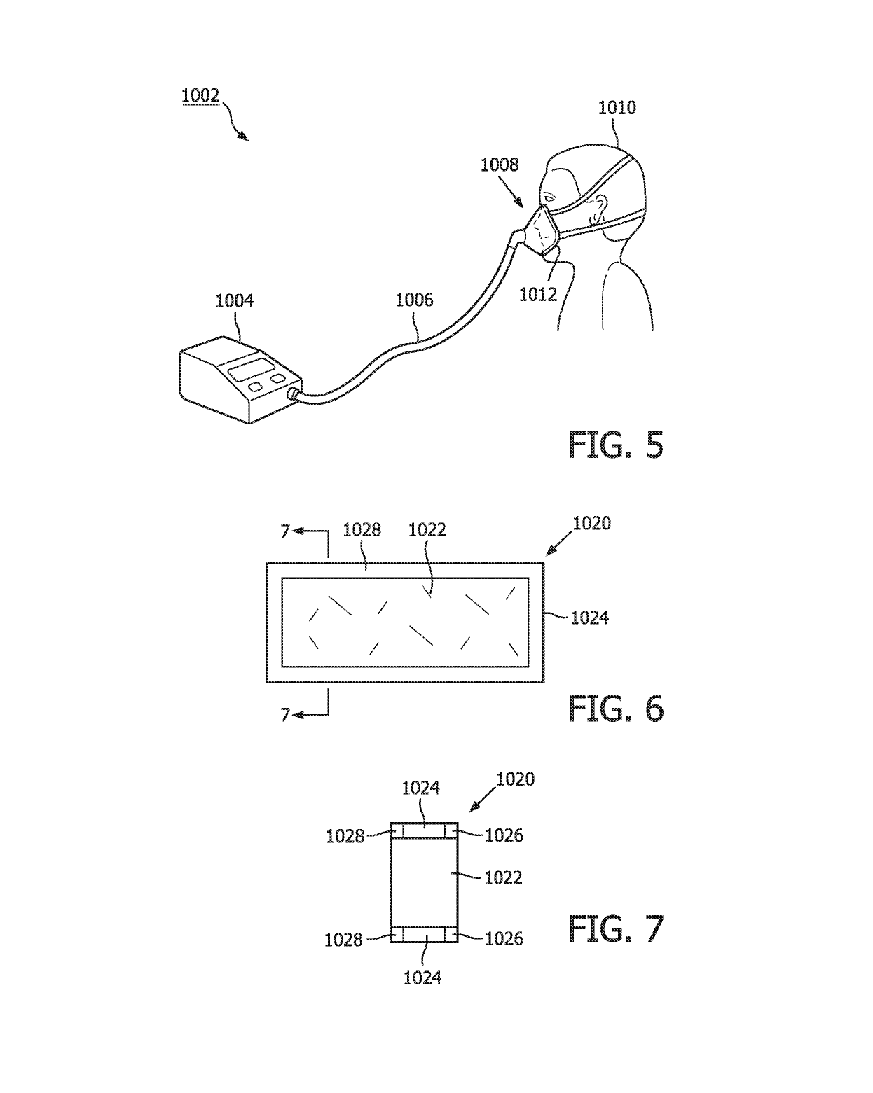

An example airway pressure support system 1002 according to one particular, non-limiting exemplary embodiment of the present invention is shown in FIG. 5. System 1002 includes a pressure/flow generator 1004, a delivery conduit 1006, a patient interface device 1008 structured to engage about an airway of the patient, and a headgear 1010 for securing patient interface device 1008 to the head of a patient (not numbered). Pressure generating device 1004 is structured to generate a flow of breathing gas which may be heated and/or humidified. Pressure generating device 1004 may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pennsylvania), and auto-titration pressure support devices. Delivery conduit 1006 is structured to communicate the flow of breathing gas from pressure generating device 1004 to patient interface device 1008. Delivery conduit 1006 and patient interface device 1008 are often collectively referred to as a patient circuit.

A BiPAP® device is a bi-level device in which the pressure provided to the patient varies with the patient's respiratory cycle, so that a higher pressure is delivered during inspiration than during expiration. An auto-titration pressure support system is a system in which the pressure varies with the condition of the patient, such as whether the patient is snoring or experiencing an apnea or hypopnea. For present purposes, pressure/flow generating device 1004 is also referred to as a gas flow generating device, because flow results when a pressure gradient is generated. The present invention contemplates that pressure/flow generating device 1004 is any conventional system for delivering a flow of gas to an airway of a patient or for elevating a pressure of gas at an airway of the patient, including the pressure support systems summarized above and non-invasive ventilation systems. Although described herein in example embodiments wherein a pressurized flow of gas is utilized, it is to be appreciated that embodiments of the invention as described herein could also be readily employed in other generally non-pressurized applications (e.g., without limitation, in high flow therapy applications).

In the exemplary embodiment, patient interface device 1008 includes a patient sealing assembly 1012, which in the illustrated embodiment is a full face mask. It is to be appreciated, however, that other types of patient sealing assemblies, such as, without limitation, a nasal/oral mask, a nasal cushion, or any other arrangements wherein rainout is a potential concern, which facilitate the delivery of the flow of breathing gas to the airway of a patient may be substituted for patient sealing assembly 1012 while remaining within the scope of the present invention. It is also to be appreciated that headgear 1010 is provided solely for exemplary purposes and that any suitable headgear arrangement may be employed without varying from the scope of the present invention.

FIGS. 6 and 7, respectively show a schematic front view, and a sectional view, of an air filtration assembly 1020 in accordance with an example embodiment of the present invention. Air filtration assembly 1020 includes a filter portion 1022 formed from a suitable filtration media (e.g., without limitation, a woven filter media) and a mounting portion 1024 disposed adjacent filter portion 1022. In the example embodiment shown in FIG. 6, mounting portion 1024 is formed generally as a frame positioned around filter portion 1022, however, it is to be appreciated that other arrangements of filter portion 1022 and mounting portion 1024 may be employed without varying from the present invention.

Mounting portion 1024 may include a compliant seal 1026 formed from a suitable compliant material (e.g., without limitation, Silicone, Flurosilicone, Fluro Elastomer, Natural Rubber Polyisoprene, Butyl, Ethlene Propylene, Nitrile) which is positioned and adapted to sealingly engage against another object, such as, for example, without limitation, a housing of a pressure generating device (e.g., without limitation, pressure generating device 1004 of FIG. 5) or another air filter. Alternatively, mounting portion 1024 may itself sealingly engage against a compliant seal provide on the other object to which air filtration assembly 1020 is disposed.

Mounting portion 1024 further includes a series of first magnetic field emission structures 1028 positioned therein or thereon which are structured to magnetically interact with a series of second magnetic field emission structures which has the same code as the first magnetic field emission structures but is a mirror image of the first magnetic field emission structures. In an example embodiment of the present invention, first magnetic field emission structures 1028 are in the form of a ferrous magnetic strip, however other forms may be employed without varying from the scope of the present invention. In example embodiments of the present invention, first magnetic field emission structures 1028 have been attached to mounting portion 1024 via one of: over-molding, heat staking and ultrasonic welding. However, it is to be appreciated that other suitable attachment methods and/or mechanisms may be employed without varying from the scope of the present invention. As another example, a magnetic plastic, may be utilized either in conjunction with another material, or solely as both mounting portion 1024 and first magnetic field emission structures 1028.

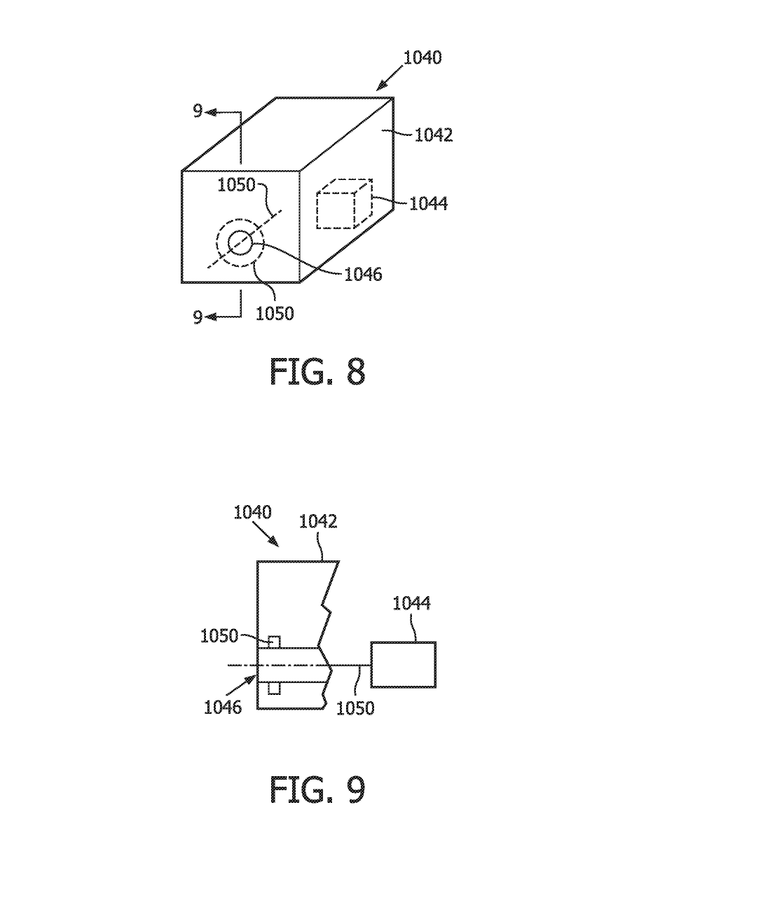

Referring now to FIGS. 8 and 9, schematic isometric and sectional views of a pressure generating device 1040 for producing a flow of a gas, similar to pressure generating device 1004 previously discussed in conjunction with FIG. 5, in accordance with an example embodiment of the present invention is shown. Pressure generating device 1040 includes a housing 1042. An air compressor 1044 for producing the flow of the gas is disposed in housing 1042. An air inlet 1046 is defined in housing 1042 and is in fluid communication with air compressor 1044 via a conduit 1050. Air inlet 1046 is structured to allow the passage of air threrethrough (e.g., from the ambient environment) to air compressor 1044 (via conduit 1050) for creating the flow of gas from pressure generating device 1040. Such flow of gas is communicated via another conduit to an outlet in housing 1042. In the example embodiment shown in FIG. 8, air inlet 1046 is generally circular in shape, however, it is to be appreciated that air inlet 1046 may be of other shapes without varying from the scope of the present invention.

Continuing to refer to FIGS. 8 and 9, housing 1042 includes a series of second magnetic field emission structures 1050 positioned therein or thereon at or about air inlet 1046. Second magnetic field emission structures 1050 are positioned and structured to interact with a corresponding first series of magnetic field emission structures associated with a filtration assembly which is disposable at air inlet 1046, such as first series of magnetic field emission structures 1028 of filtration assembly 1020 previously discussed. In an example embodiment of the present invention, second magnetic field emission structures 1050 are in the form of a ferrous magnetic strip, however other forms may be employed without varying from the scope of the present invention. In example embodiments of the present invention, second magnetic field emission structures 1050 have been attached to housing 1042 via one of: over-molding, heat staking, ultrasonic welding, hot plate welding and laser welding. However, it is to be appreciated that other suitable attachment methods and/or mechanisms may be employed without varying from the scope of the present invention.

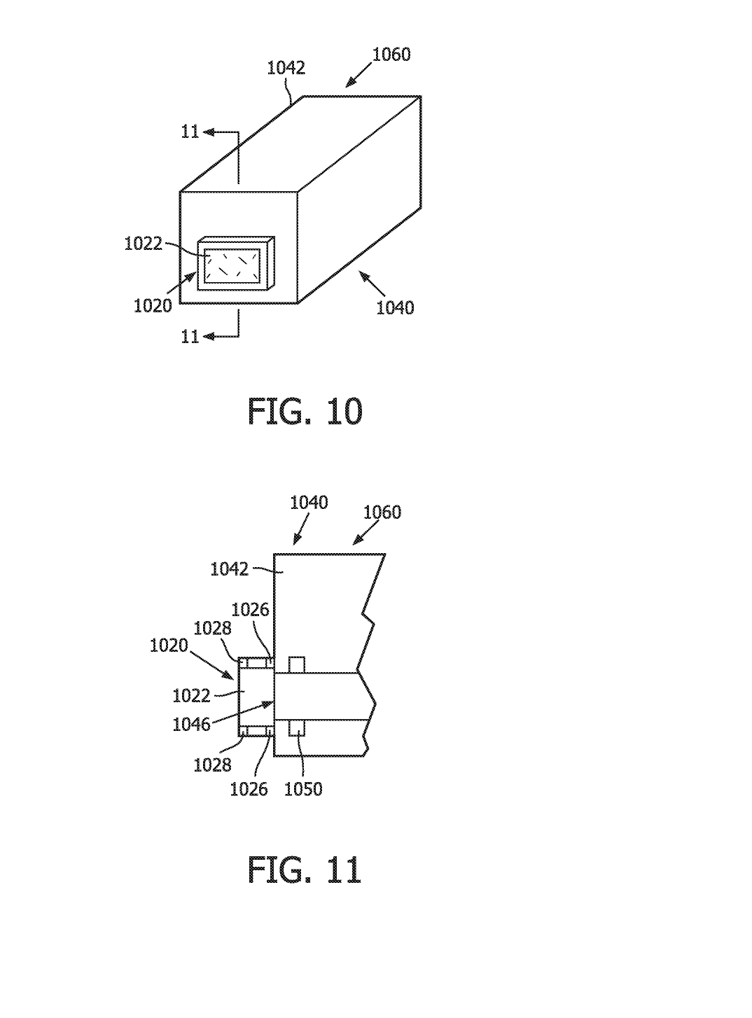

Referring now to FIGS. 10 and 11, schematic isometric and sectional views of a system 1060 for generating a flow of a gas, in accordance with an example embodiment of the present invention is shown. System 1060 includes a pressure generating device (such as pressure generating device 1040 previously discussed in regard to FIGS. 8 and 9), and an air filtration assembly (such as air filtration assembly 1020 previously discussed in regard to FIGS. 6 and 7). Air filtration assembly 1020 is coupled to housing 1042 of pressure generating device 1040 at or about air inlet 1046. In such arrangement, first magnetic field emission structures 1028 of air filtration assembly 1020 are oriented according to a code and second magnetic field emission structures 1050 of pressure generating device 1040 are oriented according to a mirror image of the code. As a result of such positioning and arrangement of first magnetic field emission structures 1028 and second magnetic field emission structures 1050, air filtration assembly 1020 is coupled to housing 1042 of pressure generating device 1040 by the magnetic attraction between first magnetic field emission structures 1028 and second magnetic field emission structures 1050. Also, as shown in the sectional view of FIG. 11, when air filtration assembly 1020 is coupled to housing 1042, compliant seal 1026 of air filtration assembly 1020 sealingly engages housing 1042, thus limiting air entering air inlet 1046 to only that which passes through filter portion 1022 of air filtration assembly 1020.

From the foregoing, it is to be appreciated that embodiments of the present invention provide numerous benefits over conventional filter/pressure generating device arrangements. As one example, only air filtration elements with the properly programmed magnetic field emission structures will attach to the pressure generating device. Accordingly, counterfeit air filtration elements and/or other improper air filtration elements cannot be used on a device employing the present invention. As another example, the magnetic fields produced by the interacting magnetic field emission structures can readily be tuned to ensure a proper seal between the air filtration element and the pressure generating device. As yet a further example, embodiments of the present invention provide for control of the installation orientation of air filtration elements. Also, multiple air filtration elements can be used simultaneously.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

What is claimed is:

1. An air filtration assembly comprising:
a filter portion formed from a filtration media; and
a mounting portion disposed adjacent the filter portion, the mounting portion having a series of first magnetic field emission structures positioned therein or thereon.

2. The air filtration assembly of claim 1, wherein the series of first magnetic field emission structures are in the form of a ferrous magnetic strip.

3. The air filtration assembly of claim 1, wherein the first magnetic field emission structures are coupled to the mounting portion via over-molding.

4. The air filtration assembly of claim 1, wherein the first magnetic field emission structures are coupled to the mounting portion via heat staking.

5. The air filtration assembly of claim 1, wherein the first magnetic field emission structures are coupled to the mounting portion via ultrasonic welding.

6. The air filtration assembly of claim 1, wherein the mounting portion is formed from a magnetic plastic which includes the first magnetic field emission structures.

7. The air filtration assembly of claim 1, wherein the mounting portion comprises a compliant seal structured and positioned to sealingly engage against another object.

8. The air filtration assembly of claim 1, wherein the mounting portion is formed generally as a frame positioned around the filter portion.

9. A pressure generating device for producing a flow of a gas, the pressure generating device comprising:
a housing;
an air compressor disposed in the housing for producing the flow of the gas; and
an air inlet defined in the housing, the air inlet in communication with the air compressor and structured to allow the passage of air therethrough to the air compressor for creating the flow of the gas,
wherein the housing comprises a series of second magnetic field emission structures positioned therein or thereon at or about the air inlet, the series of second magnetic field emission structures positioned and structured to interact with a corresponding first series of magnetic field emission structures associated with a filtration assembly which is disposable at the air inlet.

10. The pressure generating device of claim 9, wherein the series of second magnetic field emission structures are in the form of a ferrous magnetic strip.

11. A system for generating a flow of a gas, the system comprising:
a pressure generating device comprising:
a housing;
an air compressor disposed in the housing for producing the flow of the gas; and
an air inlet defined in the housing, the air inlet in communication with the air compressor and structured to allow the passage of air therethrough to the air compressor for creating the flow of the gas; and
an air filtration assembly coupled to the housing at or about the air inlet, the air filtration assembly comprising:
a filter portion formed from a filtration media; and
a mounting portion disposed adjacent the filter portion, the mounting portion having a series of first magnetic field emission structures positioned therein or thereon, the first magnetic field structures oriented according to a code,
wherein the housing comprises a series of second magnetic field emission structures positioned therein or thereon at or about the air inlet, the second magnetic field structures oriented according to a mirror image of the code, and
wherein the filtration assembly is coupled to the housing by the magnetic attraction between the first magnetic field emission structures and the second magnetic field emission structures.

12. The system of claim 11, wherein the series of first magnetic field emission structures are in the form of a ferrous magnetic strip and the series of second magnetic field emission structures are in the form of another ferrous magnetic strip.

13. The system of claim 11, further comprising a compliant seal disposed between the mounting portion and the housing of the pressure generating device.

14. The system of claim 11, wherein the mounting portion of the filtration element comprises a compliant seal sealingly engaged against the housing of the pressure generating device.
